(19) United States
(12) Patent Application Publication
(10) Pub. No.: US 2008/0027506 A1
(43) Pub. Date: Jan. 31, 2008

(54) APPARATUS AND METHOD FOR PAIN CONTROL THROUGH NERVE STIMULATION BY AN INTRA-ORAL SOURCE

(76) Inventor: John W. Cuozzo, Lebanon, NJ (US)

Correspondence Address: DILLON & YUDELL LLP, 8911 NORTH CAPITAL OF TEXAS HWY, SUITE 2110, AUSTIN, TX, 78759, US

(21) Appl. No.: 11/829,241

(22) Filed: Jul. 27, 2007

Related U.S. Application Data
(60) Provisional application No. 60/833,682, filed on Jul. 27, 2006.

(51) Int. Cl. A61N 1/34

(52) U.S. Cl. 607/47

(57) ABSTRACT

A method of pain control via the nervous system of a mammal includes contacting an oral tissue of a mammal with an energy source and imparting energy to the oral tissue to reduce perception of a pain response from the central or peripheral nervous system of the mammal.

### PRIORITY CLAIM AND CROSS-REFERENCE

This application claims priority to U.S. Patent Applicant Ser. No. 60/833,682, filed Jul. 27, 2006, and incorporates by reference U.S. Pat. No. 6,954,668.

### BACKGROUND OF THE INVENTION

1. Technical Field

The present invention relates to an apparatus and method for pain control through nerve stimulation by an intra-oral source.

2. Description of the Related Art

U.S. Pat. No. 6,954,668 describes a method and apparatus for intra-oral stimulation of the trigeminal nerve.

### SUMMARY OF THE INVENTION

The present invention is directed to pain reduction through nerve stimulation. According to one embodiment, a method of pain control via the nervous system of a mammal includes contacting an oral tissue of a mammal with an energy source and imparting energy to the oral tissue to reduce perception of a pain response from the central or peripheral nervous system of the mammal.

Additional objects, features, and advantages of the present invention will become apparent from the following detailed written description.

### BRIEF DESCRIPTION OF THE DRAWINGS

The novel features believed characteristic of the invention are set forth in the appended claims. The invention itself however, as well as a preferred mode of use, further objects and advantages thereof, will best be understood by reference to the following detailed description of one or more illustrative embodiments when read in conjunction with the accompanying drawings, wherein like reference numerals identify like or corresponding elements:

FIG. 1 depicts a sectional view of a tooth contacted by an intra-oral appliance in accordance with the present invention;

FIG. 2 illustrates an occlusal plan view of an intra-oral appliance in accordance with a preferred embodiment of the present invention, wherein line 2-2 identifies the location of the section view of FIG. 1;

FIG. 3 depicts a median sagittal section of the human brain, including a close-up schematic representation of the exit of the trigeminal nerve from the pons;

FIG. 4A illustrates a lateral partial sectional view of the human head with maxillary and mandibular teeth in normal occlusion;

FIG. 4B depicts a cross-section of the human cranium taken along line 1-1 of FIG. 4A;

FIG. 5A illustrates an occlusal plan view of a human canine tooth contacted by an intra-oral appliance in accordance with the present invention, wherein line 3-3 identifies the location of the section view of FIG. 5B; and

FIG. 5B illustrates a labio-lingual sectional view of a human canine tooth contacted by an intra-oral appliance in accordance with the present invention.

### DETAILED DESCRIPTION OF A PREFERRED EMBODIMENT

I. Theory

A. Innervation of the Periodontium

In 1957, Bernick investigated human and monkey periodontium and confirmed that there are two main groups of neural bundles coming from the periodontium: one entering at the apex of the tooth and the other entering through the cribriform plate.1 He also observed spindle-like nervous structures in the apical third of the ligament, nerve endings terminating in the cementum, and fine unmyelinated fibrils forming a network in the periodontal ligament. In 1923, Gerard described the trigeminal or fifth cranial nerve as follows: “The trigeminal nerve is composed of a large sensory division whose unipolar cells are located in the Gasserian ganglion, and a small motor division distributed entirely through the mandibular branch of the nerve. The skin of the face and the mucous membrane of the mouth, tongue, and nose are supplied by pain, tactile and thermal branches which pass into all three branches of the trigeminal nerve; these are the opthalmic, maxillary, and mandibular nerves. Sensory fibers also accompany the motor root into the brain stem, their unipolar cells of origin forming the mesencephalic nucleus, which is an unusual location for the sensory cells. These fibers are believed to supply the muscles innervated by the motor division of the trigeminal nerve. The main sensory root carries the usual cutaneous sensation.”2 Corbin and Harrison in 1940 described the trigeminal as the great cutaneous sensory nerve of the face, the sensory nerve to the mucous membranes, and other internal structures of the head. They noted that the nerve has two roots: a main sensory and a motor root, which includes, in addition to the motor fibers, proprioceptive sensory fibers from the mesencephalic nucleus.3

B. Mesencephalic Nucleus

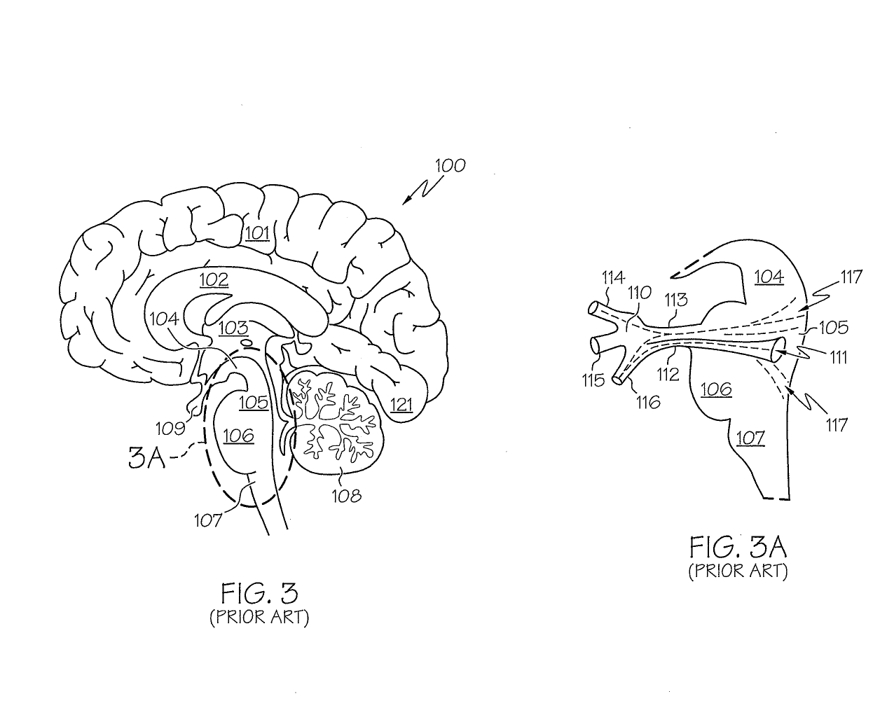

Thelander in 1924 described the mesencephalic nucleus of the trigeminal nerve as a narrow band of cells situated laterally between the central gray and the mesencephalic reticular formation of the mesencephalon (reference numeral 105 of FIG. 3). The mesencephalic nucleus extends from the posterior commisure to below the level of the trigeminal motor nucleus. The cells of the mesencephalic nucleus are predominately unipolar and have been compared to spinal ganglion cells. The similarities between the cells of the mesencephalic nucleus and the spinal ganglia as first order neurons have also been pointed out.4 The studies of Corbin and Harrison further added to the clarification of the function of the mesencephalic nucleus. They demonstrated the peripheral distribution of the fibers from this area and showed that the nucleus is activated by jaw opening movements and pressure stimulation of teeth and soft tissue in the mouth.5 Jerge discovered three types of neurons in the mesencephalic nucleus. He classified two types of dental pressoreceptors and one type of muscle proprioceptor. The first type of dental pressoreceptor represented activity from a single tooth that had been stimulated. The second type of pressoreceptor represented activity from a group of stimulated teeth and from adjacent soft tissues.6

C. Nerve Fibers in the Trigeminal Nerve of the Cat

Gerard in 1923 sectioned the trigeminal nerve of the cat, which has been found to be of similar construction and function to corresponding human nervous structures. Gerard stated: “In cross-section the nerve was seen to consist of fibers varying from the unmyelinated ones of 1.5 microns to the largest myelinated fibers of 16 microns in diameter. These latter belonged to the motor root and were always found on the dorsal surface of the nerve. There were very few of them, only 46 and 42 in two nerves counted, the majority of the motor nerves being between 10.8 and 11.7 microns in diameter.”7 He asserted that the majority of the sensory fibers in the nerve trunk ranged from 5.3 to 8 microns. Brashear found the largest fibers in the inferior alveolar nerve of the cat to be 16 microns.8 He also found the pulpal nerve fibers to only be as large as 9 microns. Windle in 1927 said, “since practically no large myelinated fibers and few unmyelinated ones pass into the pulp cavity, these must belong to the nerves innervating the periodontal membrane and gums.”9

D. Pressure Response

Duval in 1833 showed that the dentin is acutely sensitive to pain, and this remains the presently accepted view.10 Peaselee in 1857 mentioned that pressure can be detected and localized by individual teeth. He emphasized that this power of localization was due to the innervation of the periodontal ligament and was still present after removal of the pulp.11 Stewart in 1927 demonstrated that the tactile thresholds of teeth were practically unchanged after removal of their pulps. Pfaffinan in 1939 was the first person to use electrophysiologic methods to register the action potentials in the dental nerves of the cat. He recorded afferent impulses from the teeth, which were induced by graduated force applications and noxious stimuli. He again concluded (in agreement with almost everyone) that the tactile receptors are located in the periodontal ligament and enter through the cribriform plate. He believed this because the tactile responses that he recorded diminished very little after he destroyed the apical nerve coming out of the tooth by cautery.12

Contradictory evidence was presented by Loewenstein and Rathkamp in 1955 in their studies of human teeth with pulps removed. They found higher threshold values (diminished tactile responses) in pulpless teeth. In an attempt to determine the location of the dental pressoreceptors, the normal tooth to be examined was covered with a metallic crown. Pressure tests on these teeth revealed a slightly diminished response compared to normal uncrowned teeth. Vital and pulpless teeth that were covered by metal crowns showed no differences at all. These results were based upon conscious perception of stimuli by individuals rather than electrical impulses recorded from nerve trunks, which provide more accurate data. Based upon these studies, it can be concluded that pressoreceptors are removed in vigorous pulp removal extending too far through the apex of a tooth, or that the results of Loewenstein and Rathkamp were inaccurate due to subjective data collection methods, or that some sensitivity to pressoreception resides in the pulp of a tooth. Of these three possibilities, the first two appear most likely.

E. Sensory Fibers in the Inferior Alveolar Nerve of the Cat

During a physiologic investigation of the inferior alveolar nerve of the cat, diphasic action potentials resulting from tapping the incisal edge of the mandibular canine tooth with forces greater than 4 grams were recorded by means of silver electrodes attached to the inferior alveolar nerve where it exits from the mandibular foramen. If the force was maintained, higher initial spikes were observed, followed by smaller spikes having an asynchronous pattern. When the sustained force was removed, a brief high voltage discharge was occasionally observed. It was presumed that the origin of these potentials was the periodontal ligament. These potentials were superimposed on the background potentials of lesser magnitude. Increases in applied force beyond 40 grams caused only moderate increases in amplitude. It is believed that as heavier forces were applied to the tooth more nerve fibers were recruited to communicate the sensed pressure. According to Gasser in 1934,13 Ruch and Patton in 196514, Boyd in 195415, and Hunt in 195416, the lightest force first recruits the fastest nerve fibers, which are the ones with the largest diameters.

When cross-sectioned, it was found that the nerve fibers of the inferior alveolar nerve of the cat varied in diameter from 0 to 16 microns. The mean percent of the 14-16 micron fibers in the nerve sections was 2.26% of the total mean. Those between 6 and 14 microns comprised 48.56% of the total, with 49.18% of the fibers were below 6 microns. These smaller fibers are believed to be associated with the periodontal ligament pain responses. The 6 to 14 micron group comprises thermal fibers from the oral cavity, pulpal fibers, and tactile fibers from the gingiva. Relevant to the present discussion is that the tactile, mechanoreceptor or proprioceptive receptor has as its route of conduction in the largest (i.e., 14-16 micron) fibers, which are the fewest and respond to the lightest touch.

F. Tooth as Piezoelectric Conductor

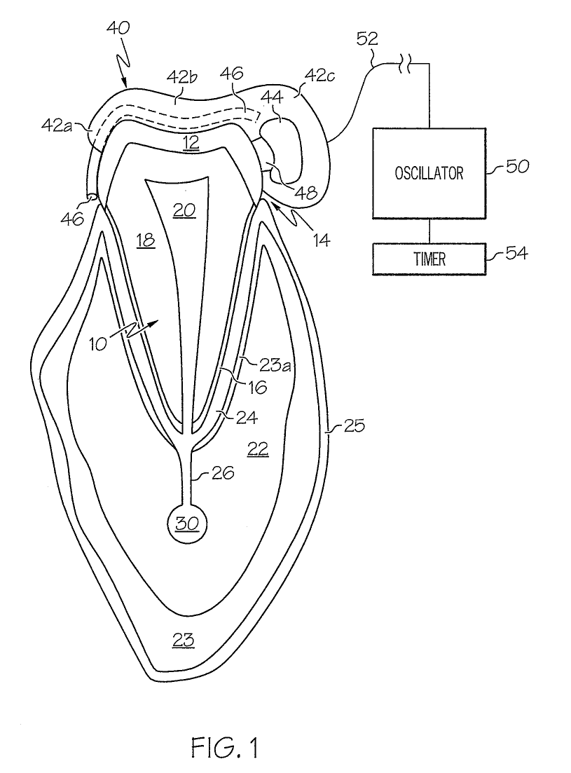

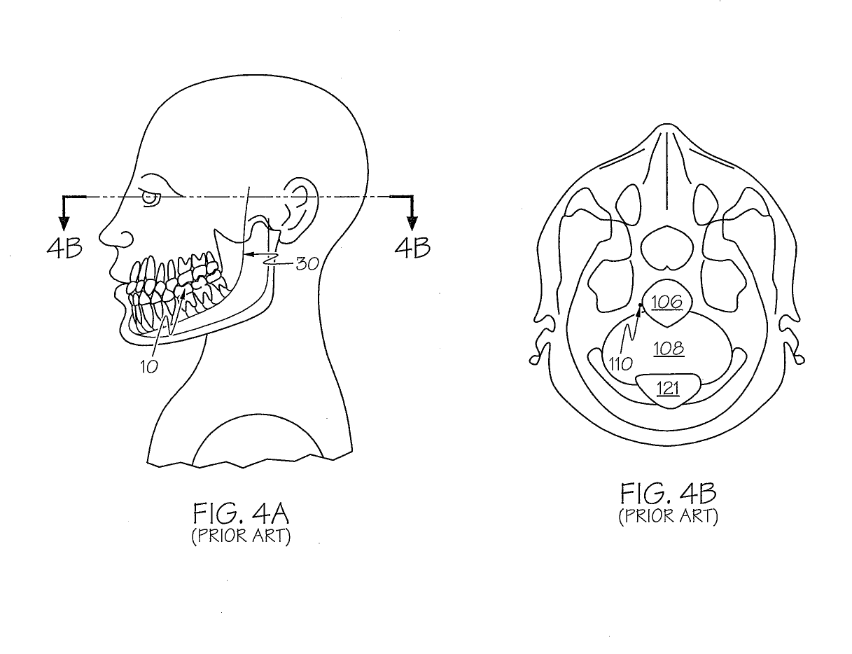

The mandibular first molar tooth 10 is depicted within the lateral partial sectional view of the human head provided in FIG. 4A. As illustrated in the cross-sectional buccolingual view of the mandibular first molar provided in FIG. 1, every human tooth 10 is a composite structure formed of different materials. The exposed surface of tooth 10 is covered with enamel 12 to gumline 14 or even slightly below gumline 14. Below gumline 14, the surface of the root(s) of tooth 10 is covered with cementum 16. (Only the distal root of the mandibular first molar is shown in FIG. 1.) The interior of the tooth 10 underneath enamel 12 and cementum 16 is formed of dentin 18. Finally, in the interior cavity of tooth 10 is pulp 20, which includes the pulpal nerves.

The root of tooth 10 is anchored in mandibular cancellous bone 22 and cribriform plate 23a within an outer cortical plate 23 by periodontal ligament 24. Periodontal ligament 24 is attached to a nerve bundle 26, which receives nerve fibers from both periodontal ligament 24 and pulp 20 and conducts nerve impulses to the mandibular branch of the trigeminal nerve 30 (which is also depicted at reference numeral 30 of FIG. 4A and at reference numeral 116 of FIG. 3). Layers of connective tissue/membrane 25 cover the lamellated cortical plate 23.

When enamel 12 first develops, enamel 12 is formed of tightly packed columns or “matrix” of a relatively soft fibrous material like that of tendons and ligaments. During subsequent human development, minerals (almost exclusively calcium and phosphorous) bond within the matrix to form hydroxyapaptite crystals, also called “apatites.” These mineral crystals harden within the matrix to form a combination of hard and soft materials called the enamel “prism” or “rod,” which extends from the dentin interface to at or near the outermost surface of enamel 12. At the completion of development, enamel 12 is formed of approximately 95-97% minerals by weight, which makes enamel 12 very hard.

Athenstaedt in 1971 discovered that pressure stimulation of a tooth produces a piezoelectric effect. He found that even if you slice a tooth into thin cross-sections horizontally, positive and negative charges are elicited with pressure stimulation.17 He wrote that, “Under the effect of compression, a complete tooth has a positive charge at the occlusal surface and a negative electric charge at the root apex (piezoelectric effect).”18

II. Application of Theory

By synthesizing the foregoing information, the present invention recognizes that the pressoreceptors within periodontal ligament 24 and therefore trigeminal nerve 30 (see also FIG. 4A) can be stimulated by application of an external energy source to enamel 12. The enamel prisms first resonate when energy is applied, for example, by a mechanical, sonic or electromagnetic energy source. Because of the hardness of the hydroxyapaptite crystals of enamel 12 and the size of the crown of tooth 10, tooth 10 has a high resonance Q.19 As a result, tooth 10 is frequency-selective and is slow to respond to a driving signal, but sustains its activity for some time after an interval of forced oscillation.

The prisms of enamel 12 conduct the energy to dentin 18 and cementum 16. Dentin 18 and cementum 16 act as plates of a piezoelectric speaker, conducting the resonance via cementum 16 into periodontal ligament 24. The pressoreceptor nerve endings within nerve bundle 26 that are stimulated by the induced resonance are the largest ones (about 2-3% of the total bundle), which conduct the lightest forces. These nerve endings conduct the stimulation to the mandibular branch of the trigeminal nerve 30, thus providing a direct route to the midbrain. By stimulating the midbrain in this fashion, delta brainwaves can be induced, leading to relaxation and/or sleep.

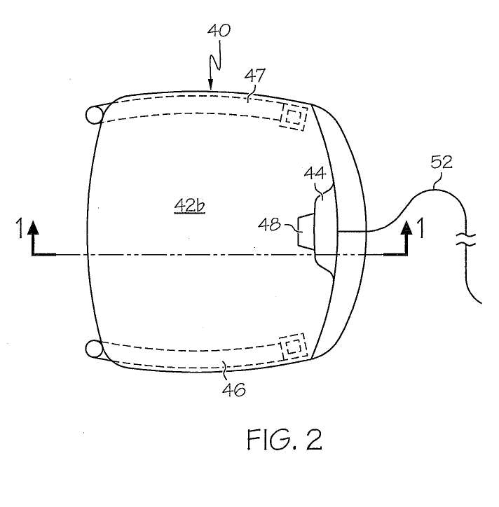

In accordance with a preferred embodiment of the present invention and as illustrated in FIGS. 1 and 2, energy is applied to tooth 10 to stimulate the trigeminal nerve by a removable and reinstallable intra-oral appliance 40. Appliance 40 includes an energy source 44 and an attachment portion 42 that removably secures energy source 44 in close contact with enamel 12 (or if tooth 10 has an artificial crown, to the artificial crown).

In the depicted embodiment, attachment portion 40 includes a first leg 42a, a slightly longer second leg 42c, and a bridge portion 42b spanning the occlusal surface of tooth 10 (in this exemplary embodiment, a mandibular tooth) to link first and second legs 42a and 42c. As illustrated in FIG. 1 in phantom, bridge portion 42b includes an embedded standard 0.040-inch diameter stainless steel orthodontic wire 46. As depicted in the occlusal plan view provided in FIG. 2, bridge portion 42b also includes a similar second embedded wire 47. When appliance 40 is installed, wires 46 and 47 respectively engage the distal and mesial inferior lingual surfaces of the crown of tooth 10 below the widest portion of the crown. Attachment portion 40 is thus removably retained on tooth 10 by the spring force of wires 46 and 47 and interference fit of the contact portion 48 of energy source 44 with the buccal or labial side of tooth 10.

Attachment portion 40 is preferably fabricated by a dental health professional from lightly cured or self-cured acrylic or other durable non-toxic material to ensure a proper fit that permits easy installation, removal, and reinstallation of appliance 40. Because the muscles of mastication (except external ptyergoid M) send proprioceptive nerve fibers into the mesencephalic nucleus, the stretching of these muscles much beyond their resting length will send interfering impulses into the same nerve nucleus that appliance 40 stimulates. The stimulation of these muscles with appliance 40 installed should therefore be minimized or avoided by sizing appliance 40 so that the bite is not opened a great deal with appliance 40 installed.

As noted above, energy source 44 can be implemented as a mechanical, sonic or electromagnetic energy source. In a typical implementation, energy source 44 is a transducer coupled by electrical conductors 52 to an external power source, such as oscillator 50. For example, in one exemplary embodiment, energy source 44 comprises an electromagnetic coil, such as commonly found in acoustic earphone speakers, and oscillator 50 comprises a portable electronic device (e.g., audio cassette player, CD player, MP3 player, etc.) that outputs low voltage analog audio frequency electrical signals via electrical conductors 52. These electrical signals are converted by the electromagnetic coil into a time-varying magnetic field that, due to the piezoelectric properties of tooth 10 discussed above, stimulates trigeminal nerve 30. In an alternative embodiment, energy source 44 can be implemented as a mechanical vibrator that vibrates at a frequency determined by an input signal received from oscillator 56.

In each of these possible embodiments, it is preferable, though not required, for the frequency range of electrical signals output by oscillator 50 to be calibrated to the resonant frequency of the specific tooth 10 on which appliance 40 is to be installed in order to achieve the maximal effect in the pressoreceptors, trigeminal nerve fibers and brain stem. It has also been found helpful to vary the amplitude and frequency of the stimulation provided by energy source 44 between uses.

In use, one or more teeth can be employed to stimulate the trigeminal nerve at a time. It is preferable, however, that only one side of the dental arch is employed at a time because impulses entering the main nerve trunk from opposite sides of the inferior or superior alveolar branches of the trigeminal nerve will tend to block each other. In addition, the effectiveness of appliance 40 is improved if use is limited in duration (e.g., approximately 30 minutes) because the wake cycle in the reticular formation of the brain is triggered if stimulation of the trigeminal nerve continues after delta waves characteristic of deep relaxation or sleep have been induced. Thus, the best response results from a metered dose, which will depend on the size of the tooth, number of teeth employed, and the individual's sleep habits and therapeutic history (e.g., previous drug therapies). Accordingly, it is useful if oscillator 50 has an associated timing mechanism, such as timer 54, to conveniently meter the duration of use. In experimentation, it has also been found helpful to alternate sides of the dental arch (e.g., alternating between the right and left first mandibular molars) approximately every seven days because the affected nerves appear to adapt to the stimulus over a period of about a week. Alternating sides, which would preferably entail the alternating use of different appliances 40 for the right and left sides, enhances the effectiveness of the therapy and maintains a high level of response on both sides of the dental arch.

As has been described, the present invention provides an intra-oral appliance for stimulating an alveolar branch of the trigeminal nerve through the pressoreceptors of one or more teeth. By doing so, relaxation and/or sleep can be induced and/or enhanced. Other beneficial applications are also contemplated. For example, stimulating the pressoreceptors of the teeth has been found to inhibit jaw muscle activity by relaxing the elevator jaw muscles (temporalis, internal ptyergoid and masseter).20 Consequently, the appliance of the present invention has application to patients with TMJ (temporomandibular joint) problems and/or bruxism (i.e., teeth grinding).

The applications of the intra-oral appliance of the present invention also include those previously addressed by direct electrical stimulation of the trigeminal nerve as taught by Zabara in U.S. Pat. No. 5,540,734, the pertinent parts of which are incorporated herein by reference. These additional applications are within the scope of the present invention.

Although the present invention and that of Zabara have common uses, several significant differences between Zabara and the present invention should be noted. First, Zabara's technique can lead to the contraction of the muscles of mastication, unlike the present invention, which relaxes them. One site for electrode placement recommended by Zabara is the mandibular (third) division 116 of the trigeminal nerve. In contrast to the ophthalmic and maxillary divisions 114-115 of the trigeminal (see FIG. 3), which carry only sensory impulses, mandibular division 116 carries both motor and sensory impulses, including motor impulses for the main muscles of mastication (e.g., masseters, internal and external pterygoids and temporal muscles). The placement of an electrode on the motor nerves that innervate the mastication muscles as taught by Zabara will send electrical impulses down the nerve to the mastication muscles as well as up the nerve to mesencephalon 105, resulting in (possibly uncomfortable) contraction of the muscles of mastication. As noted above, the present invention, by contrast, relaxes the muscles of mastication.21

Second, and more importantly, Zabara's technique is surgically invasive to the midbrain and requires direct nerve contact, while the present invention is completely non-invasive and does not utilize any direct nerve contact. As noted above, while Zabara teaches the use of either an internal or external neurostimulator (electrical signal generator), Zabara's electrodes are always attached directly to the afferents of the trigeminal and/or glossopharyngeal nerves, requiring surgical implantation of the electrodes within the patient's cranial cavity. The present invention, by contrast, can be practiced completely non-invasively without any direct nerve contact. Consequently, oral and cerebral tissues remain intact and undisturbed, reducing the risk of side effects and complications (e.g., infection).

In addition, an intra-oral appliance in accordance with the present invention can be utilized to diminish pain perception in mammals. As is well known in the art, a mammalian nervous system (including the human nervous system) includes a central nervous system, which for convenience is described as comprising the brain (encephalon) and the spinal cord (medulla spinalis)²², and a peripheral nervous system, connected to the central nervous system, that is distributed in various tissues of the body. Pain is experienced when a pain stimulus stimulates pain receptors, which signal occurrence of the pain stimulus via specialized peripheral nerves to the spinal cord and ultimately to the brain. The brain processes the pain stimulus and may transmit impulses via appropriate nerves to cause the body to react to the pain stimulus.

Research from 1987 showed a connection between the rostral brain stem (area of entry of the trigeminal nerve to the midbrain) and the median nerve of the hand.²³ Earlier research from 1983 linked the transmission of somatosensory evoked potentials (SEPs) to the caudal brainstem (which is the origin of the trigeminal nuclei) and not the rostral.²⁴ This result agrees with another study of the same year.²⁵ SEPs evoked by stimulation of the median nerve and peroneal nerve can be recorded from the scalp.²⁶ Based upon such research and empirical results of testing the intra-oral appliance of the present invention, the connections between the central nervous system and the peripheral nervous system provide signaling pathways that can be evoked for transmission of stimuli in either direction.

According to the present invention, pain in a peripheral region of the body (e.g., head, hand, leg, etc.) can be controlled through intra-oral nerve stimulation, as described herein. Control of the pain can be as effective as a complete pain “block.”

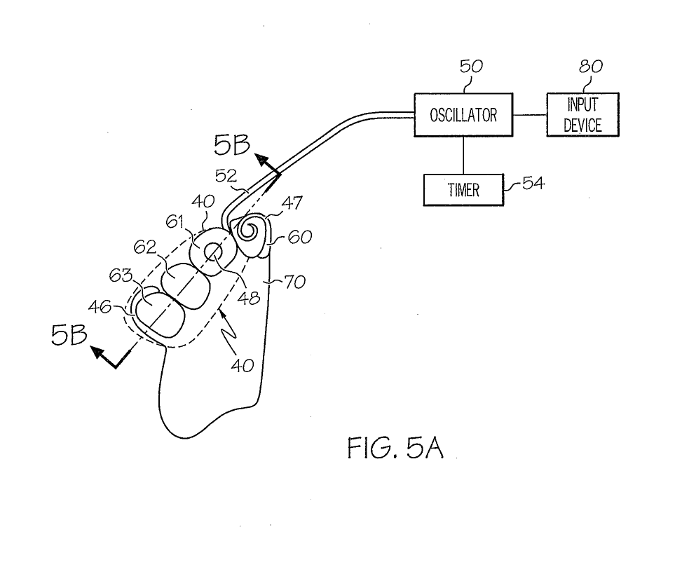

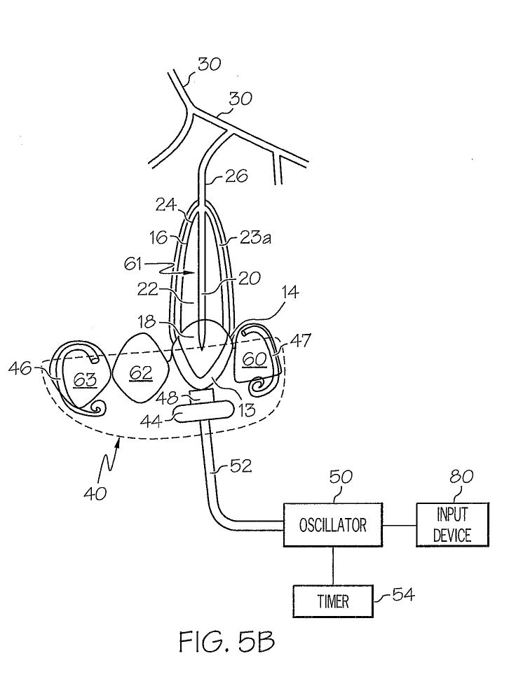

With reference now to FIGS. 5A and 5B, there are respectively illustrated an occlusal plan view and a labio-lingual sectional view of a human maxillary canine tooth contacted by an intra-oral appliance 40 in accordance with the present invention. Line 3-3 in FIG. 5A identifies the location of the section view of FIG. 5B.

In the occlusal view of FIG. 5A, the maxilla palatal process 70 and teeth including right maxillary lateral incisor 60, right maxillary cuspid (or canine) 61, right maxillary first bicuspid 62 and right maxillary second bicuspid 63 are depicted. An intra-oral appliance 40, substantially as previously described but physically reconfigured for different oral placement, is removably installed by wires 46, 47 engaging right maxillary lateral incisor 60 and right maxillary second bicuspid 63. As installed, contact portion 48 of intra-oral appliance preferably contacts the incisal edge or “tip” of the enamel edge of right maxillary cuspid 61, which as described in the 1966 Cuozzo thesis elicits the optimal response from the piezoelectrically stimulated tooth and the periodontal ligament. However, if occlusal imbalances preclude such placement or an alternative placement is simply desired, placement on a labial-incisal, lingual insical, inter-proximal, or other tooth surface can alternatively be used. As best seen in FIG. 5B, nerve bundle 26 of right maxillary cuspid 61 conducts nerve impulses to trigeminal nerve 30 (in this case, its anterior superior maxillary division).

In experimental testing utilizing the arrangement shown in FIGS. 5A-5B, trigeminal nerve 30 was stimulated with oscillator 50 producing sine waves having a voltage of less than one volt, and more particularly, about 44.4 mV, and having an average frequency of between 1.0-2.5 kHz, and more particularly about 1.89 kHz, and an EMF of between 0.75-1.5 mT, and more particularly, between 1.0-1.2 mT. Under these conditions, pain fiber transmission and pain perception from a carpal tunnel/arthritic condition was blocked from the subject's right hand after 15 minutes of continuous stimulation. When the trigeminal stimulation was discontinued, perception of the pain from the subject's right hand returned after a few minutes. The experiment was successfully repeated utilizing a higher EMF of between 2.8-4.8 mT, and more particularly, between 3.0-3.7 mT.

Thus, experimental testing verifies that pain perception arising from pain stimuli arising in the peripheral nervous system, including without limitation, the median and/or radial and/or ulnar nerves of the upper right extremity, can be effectively controlled by intra-oral nerve stimulation. In general, experimental testing has not shown any of the selected waveform (sine wave, sawtooth, etc.), the voltage swing, the frequency or the EMF to be critical factors in achieving diminution in pain perception. However, research such as that documented in the 1966 Cuozzo thesis indicates that greater nerve stimulation and therefore greater reduction in subjective pain perception is achieved if the frequency is tuned to the harmonic frequency of the tooth (or other oral tissue) to which energy is applied by intra-oral appliance 40. Consequently, in at least some embodiments, it is desirable if oscillator 50 has a selectable frequency or range of frequencies that can be selected by the user or a fabricator of intra-oral appliance 40 in accordance with the tooth to which energy is applied by intra-oral appliance 40.

In order to continue the pain reduction achieved through intra-oral nerve stimulation, the apparatus is preferably configured to selectively operate in one or more modes allowing extended stimulation of the nerve. For example, in one embodiment, timer 54 is programmed to cause oscillator 50 to discontinue output for brief periods (e.g., between 5-15 minutes each hour) and then resume output. This discontinuity in stimulation lessens the reduction in the nerve response attributable to continuous stimulation. In addition, in at least some embodiments, nerve adaptation is counteracted by configuring oscillator 50 through hardware, software or a combination of hardware and software to automatically increase EMF during stimulation (e.g., stepping up at regular intervals or continuously during a stimulation period) or pulse ENF during stimulation (e.g., alternate between a high EMF of 4.8 mT and a low EMF of 0.5 mT) to compensate for nerve adaptation.

In a further embodiment of the present invention, the apparatus is configurable to receive pain level feedback from a user in order to tailor its response to nerve adaptation to that particular user. For example, in one embodiment, oscillator 50 may be configured in hardware and/or software with a longer period of cessation between periods of stimulation (e.g., 15-30 minutes) and, in response to a user providing an indication of pain (i.e., a user input entered via optional input device 80), decrease the period of cessation between periods of stimulation. Alternatively, in an embodiment having variable EMF, in response to a user providing an indication of pain, oscillator 50 may be configured in hardware and/or software to increase the minimum EMF (e.g., from 0.5 mT to 0.75 mT) or maximum EMF (e.g., 1.5 mT to 5.0 mT).

In yet another application, intra-oral appliance 40 can be utilized to reduce edema in vessels and tissues of the nasal mucosa served by the sensory division of the trigeminal nerve in accordance with the technique described above.

In still another application, intra-oral appliance 40 can be utilized to reduce migraine headache pain originating in the central nervous system by stimulating the trigeminal nerve in accordance with the technique described above.

While the invention has been particularly shown and described with reference to a preferred embodiment, it will be understood by those skilled in the art that various changes in form and detail may be made therein without departing from the spirit and scope of the invention. Without restricting the generality of the foregoing, alternative embodiments of the present invention may employ different designs of the intra-oral attachment portion, including “caps” covering multiple teeth. In addition, energy source 44 can be embedded in any oral appliance, including any (1) bite splint, nightguard, mouthguard, or bruxism appliance, (2) TMJ appliance (3) orthodontic brace, bonded bracket or bonded attachment, (4) orthodontic or other type of retainer, or (5) bionator. Furthermore, although certain embodiments (such as that illustrated in FIG. 1) depict an oscillator 50 and timer 54 separate from the attachment portion 40 and connected thereto by wires 52, one or both of the oscillator and timer may be integral to the attachment portion and external components, if any, may communicate with the intra-oral components via wireless (e.g., RF) signals. Moreover, although the present invention has been described with reference to embodiments in which the first mandibular molar and cuspid are employed, the scope of the present invention encompasses the stimulation of the trigeminal nerve through (1) any tooth or any combination of teeth to which neurostimulation can be applied, (2) any artificial or natural implant in the alveolar or basal bone (maxilla or mandible) that may indirectly affect the inferior or superior alveolar nerves, (3) full or partial dentures or dental bridges that may indirectly affect the inferior or superior alveolar nerves, (4) intra-oral soft tissues, and (5) tongue.

In alternative embodiments of the present invention in which a dental implant, dentures or a bridge is employed to stimulate the trigeminal nerve, an attachment portion similar to that illustrated in FIGS. 1-2 and 5A-5B, but modified to attach to the selected oral structure, can be employed. However, if stimulation is applied directly to intra-oral soft tissues, the intra-oral transducer need not be packaged in an attachment portion, but can instead be maintained in place manually or by the patient's bite.

1. A method of pain control via the nervous system of a mammal, said method comprising:
contacting an oral tissue of a mammal with a contact portion of an intra-oral appliance;
imparting energy to the oral tissue utilizing the intra-oral appliance in order to stimulate the trigeminal nerve and reduce perception of pain from the nervous system of the mammal;
receiving an input indicative of pain in the mammal; and
in response to receipt of the input, increasing energy imparted to the oral tissue by the intra-oral appliance.

2. The method of claim 1, wherein the oral tissue includes at least one tooth.

3. The method of claim 2, wherein:
the at least one tooth includes an incisal edge; and
said contacting comprises contacting the incisal edge of the at least one tooth with the energy source.

4. The method of claim 1, wherein imparting energy to the tooth includes imparting energy at a frequency of at least approximately 1.0 kHz.

5. The method of claim 1, wherein imparting energy to the oral tissue to reduce perception of pain comprises imparting energy to the oral tissue to reduce perception of pain response in the head of the mammal.

6. The method of claim 1, wherein imparting energy to the oral tissue to reduce perception of pain comprises imparting energy to the oral tissue to reduce perception of pain response in an extremity of the mammal.

7. The method of claim 1, wherein imparting energy to the oral tissue further comprises imparting energy to the oral tissue to reduce edema in nasal mucosa of the mammal.

8. A method of pain control via the nervous system of a mammal, said method comprising:
contacting an oral tissue of a mammal with a contact portion of an intra-oral appliance;
imparting energy to the oral tissue utilizing the intra-oral appliance in order to stimulate the trigeminal nerve and reduce perception of pain from the nervous system of the mammal, wherein said imparting energy includes alternating first periods of imparting more energy to the oral tissue with at least one second period of imparting less energy to the oral tissue.

9. The method of claim 8, and further comprising:
in the at least one second period of imparting less energy to the oral tissue, imparting no energy to the oral tissue.

10. The method of claim 8, wherein the oral tissue includes at least one tooth.

11. The method of claim 10, wherein:
the at least one tooth includes an incisal edge; and
said contacting comprises contacting the incisal edge of the at least one tooth with the energy source.

12. The method of claim 8, wherein imparting energy to the tooth includes imparting energy at a frequency of at least approximately 1.0 kHz.

13. The method of claim 8, wherein imparting energy to the oral tissue to reduce perception of pain comprises imparting energy to the oral tissue to reduce perception of pain response in the head of the mammal.

14. The method of claim 8, wherein imparting energy to the oral tissue to reduce perception of pain comprises imparting energy to the oral tissue to reduce perception of pain response in an extremity of the mammal.

15. The method of claim 8, wherein imparting energy to the oral tissue further comprises imparting energy to the oral tissue to reduce edema in nasal mucosa of the mammal.

16. A method of pain control via the nervous system of a mammal, said method comprising:
contacting an oral tissue of a mammal with a contact portion of an intra-oral appliance;
imparting energy to the oral tissue utilizing the intra-oral appliance in response to a time-varying signal in order to stimulate the trigeminal nerve and reduce perception of pain from the nervous system of the mammal;
selecting a frequency for the time-varying signal, wherein said selecting includes:
selecting a first frequency for the time-varying signal if a first oral tissue is contacted by the contact portion; and
selecting a second frequency for the time-varying signal if a second oral tissue is contacted by the contact portion.

17. The method of claim 16, wherein the first oral tissue includes a first tooth and the second oral tissue includes a different second tooth.

18. The method of claim 16, wherein said selecting comprises setting a frequency of the time-varying signal at a signal source of said time-varying signal.

19. A method of pain control via the nervous system of a mammal, said method comprising:
contacting an oral tissue of a mammal with an energy source;
stimulating the trigeminal nerve to reduce perception of a pain response originating from a pain stimulus outside of the mouth;
receiving an input indicative of pain in the mammal; and
in response to receipt of the input, varying stimulation of the trigeminal nerve.
